(19) United States
(12) Patent Application Publication
(10) Pub. No.: US 2015/0053204 A1
(43) Pub. Date: Feb. 26, 2015

(54) PATIENT INTERFACE ASSEMBLY

(71) Applicant: KONINKLIJKE PHILIPS N.V., EINDHOVEN (DE)

(72) Inventors: Robert William Baiko, Pittsburgh, PA (US); Daniel James Miller, Cranberry Township, PA (US); Alicia Marie Brentzel, Jeannette, PA (US)

(21) Appl. No.: 14/387,940

(22) PCT Filed: Mar. 25, 2013
(86) PCT No.: PCT/IB2013/052358

§ 371 Date: Sep. 25, 2014

Related U.S. Application Data
(60) Provisional application No. 61/617,128, filed on Mar. 29, 2012.

(51) Int. Cl. A61M 16/06; A61M 16/08

(52) U.S. Cl. CPC A61M 16/0683; A61M 16/0622; A61M 16/0816
USPC 128/201.23

(57) ABSTRACT

A patient interface assembly comprises a patient interface device for communicating a gas to a patient and headgear comprising a strap arrangement for securing the patient interface device against the face of the patient. A hair grip device (30) is provided for coupling the headgear to the hair of the patient.

### FIELD OF THE INVENTION

The present invention relates to patient interfaces assemblies for transporting a gas to and/or from an airway of a user.

### BACKGROUND OF THE INVENTION

There are numerous situations where it is necessary or desirable to deliver a flow of breathing gas non-invasively to the airway of a patient, i.e. without inserting a tube into the airway of the patient or surgically inserting a tracheal tube in their oesophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnoea syndrome, in particular, obstructive sleep apnoea (OSA).

Non-invasive ventilation and pressure support therapies involve the placement of a patient interface device including a mask component on the face of a patient. The mask component may be, without limitation, a nasal mask that covers the patient's nose, a nasal pillow/cushion having nasal prongs that are received within the patient's nostrils, a nasal/oral mask that covers the nose and mouth, or a full face mask that covers the patient's face. The patient interface device interfaces between the ventilator or pressure support device and the airway of the patient, so that a flow of breathing gas can be delivered from the pressure/flow generating device to the airway of the patient.

Such devices are typically maintained on the face of a patient by headgear having one or more straps adapted to fit over/around the patient's head.

FIG. 1 shows a typical system to provide respiratory therapy to a patient. This system will be referred to in the description and claims as a “patient interface assembly”.

The system 2 includes a pressure generating device 4, a delivery conduit 16 coupled to an elbow connector 18, and a patient interface device 10. The pressure generating device 4 is structured to generate a flow of breathing gas and may include, without limitation, ventilators, constant pressure support devices (such as a continuous positive airway pressure device, or CPAP device), variable pressure devices, and auto-titration pressure support devices.

Delivery conduit 16 communicates the flow of breathing gas from pressure generating device 4 to patient interface device 10 through the elbow connector 18. The delivery conduit 16, elbow connector 18 and patient interface device 10 are often collectively referred to as a patient circuit.

The patient interface device includes a mask 12 in the form of a shell 15 and cushion 14, which in the exemplary embodiment is nasal and oral mask. However, any type of mask, such as a nasal-only mask, a nasal pillow/cushion or a full face mask, which facilitates the delivery of the flow of breathing gas to the airway of a patient, may be used as mask. The cushion 14 is made of a soft, flexible material, such as, without limitation, silicone, an appropriately soft thermoplastic elastomer, a closed cell foam, or any combination of such materials.

An opening in the shell 15, to which elbow connector 18 is coupled, allows the flow of breathing gas from pressure generating device 4 to be communicated to an interior space defined by the shell 15 and cushion 14, and then to the airway of a patient.

The patient interface assembly 10 also includes a headgear component 19, which in the illustrated embodiment is a two-point headgear. Headgear component 19 includes a first and a second strap 20, each of which is structured to be positioned on the side of the face of the patient above the patient's ear. The headgear component can however have a single strap around the back of the patient's head, or it can have two strap—one over the top and one behind—or it can have other strap configurations.

Headgear component 19 further includes a first and a second mask attachment element 22 to couple the end of one of the straps 20 to the respective side of mask 12.

A problem with this type of assembly can be that the headgear straps move as a result of movement of the patient during sleep. The movement of the straps can result in the patient interface device becoming loose or coming off.

### SUMMARY OF THE INVENTION

According to the invention, there is provided a device as claimed in the independent claims.

In one aspect, the invention provides a patient interface assembly, comprising:
a patient interface device for communicating a gas to the nose and/or mouth of a patient;
headgear comprising a strap arrangement for securing the patient interface device against the face of the patient and having at least one strap; and
a hair grip device for coupling the headgear to the hair of the patient.

This arrangement provides a coupling between the headgear and the patient's hair, and this provides a comfortable way to reduce slippage of the headgear during sleeping.

The hair grip device is for example for coupling the at least one strap of the of the strap arrangement to the hair of the patient.

The hair grip device can be fixed to the headgear (e.g. the at least one strap) so that it is an integral part of the headgear.

Alternatively, the hair grip device can comprise a releasable coupling for releasably coupling the hair grip device to the headgear. This enables the hair grip device to be attached to the hair before the headgear is applied, and this can be an easier fitting process.

The releasable coupling can then comprise a magnetic element and the at least one strap is provided with a corresponding magnetic element. In this way, the hair grip device is fitted, and the headgear is magnetically coupled to it. The magnetic element of the headgear can be fixed in position on the headgear or else it can be removably mounted. The latter arrangement enables more comfortable matching between the headgear position and the hair grip position. This applies to other types of coupling as well, not only to the magnetic coupling.

The hair grip device can for example comprise a hair comb. This is like a normal comb but typically has longer and fewer teeth which are larger and more separated than a normal comb.

The invention also provides a hair grip device for a patient interface assembly for communicating a gas to the nose and/or the mouth of a patient and headgear comprising a strap arrangement for securing the patient interface device against the face of the patient and having at least one strap,
wherein the hair grip device comprises a releasable coupling for releasably coupling the hair grip device to the headgear.

The invention also provides a headgear assembly for securing a patient interface to a patient's head, the headgear assembly being arranged to receive a hair gripping device according to the invention. The headgear assembly may comprise coupling means for coupling the headgear assembly to the hair gripping device. The headgear assembly may comprise the hair gripping device.

### BRIEF DESCRIPTION OF THE DRAWINGS

Examples of the invention will now be described in detail with reference to the accompanying drawings, in which:

FIG. 1 shows a known patient interface;

FIG. 2 shows a first example of hair grip device used the assembly of the invention;

FIG. 3 shows in rear view the head of a patient with the assembly of the invention in place;

FIG. 4 shows one example of magnetic coupling for the headgear strap; and

FIG. 5 shows another example of magnetic coupling for the headgear strap.

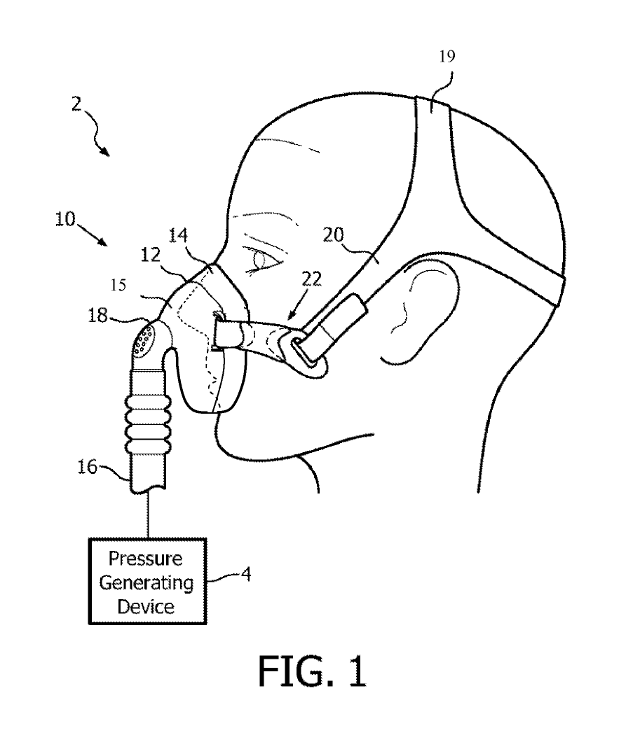

### DETAILED DESCRIPTION OF THE EMBODIMENTS

The invention provides a patient interface assembly, comprising a patient interface device for communicating a gas to a patient and headgear comprising a strap arrangement for securing the patient interface device against the face of the patient. A hair grip device is provided for coupling the headgear to the hair of the patient.

The hair grip device can be a separate item or it can be an integral part of the headgear.

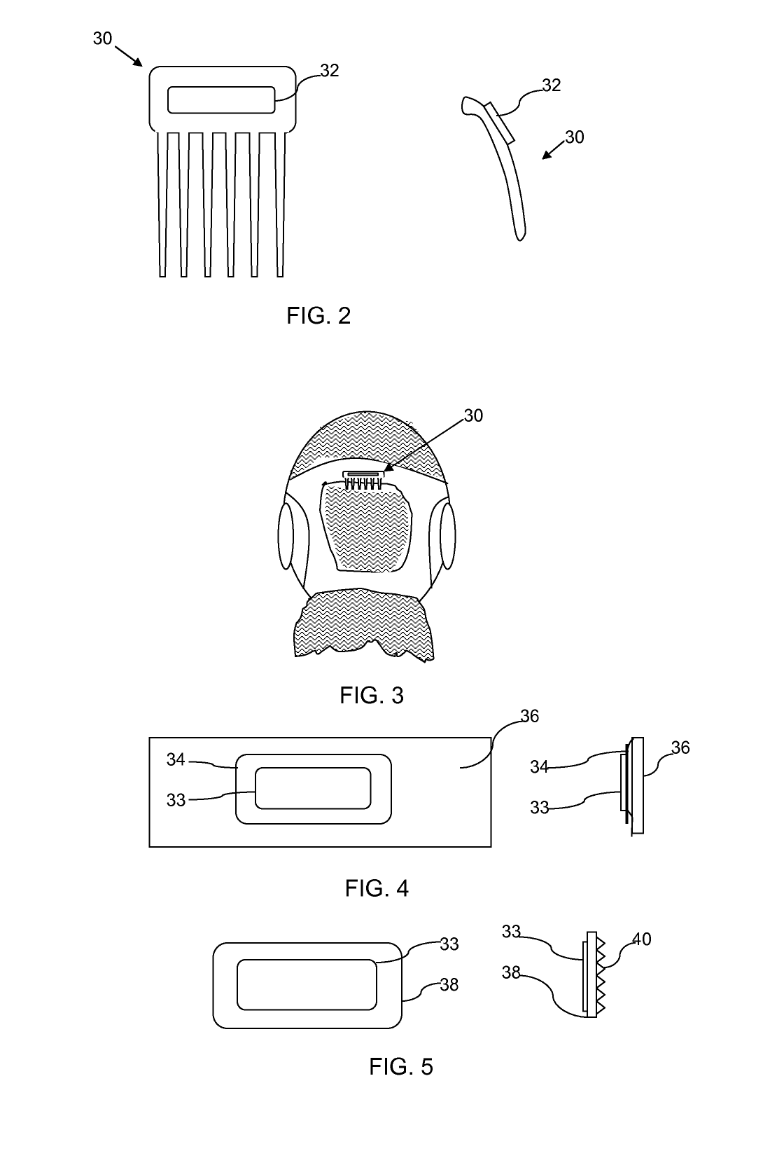

FIG. 2 shows (in plan and side view) a hair grip device in the form of a hair comb 30 for example made of plastic, with a magnet 32 fixed to the surface. Alternatively the hair comb can be entirely formed of metal. The hair comb 30 is inserted into the hair and held in position, with the magnet facing outwardly.

The headgear includes a magnet 33 associated with one of the headgear straps. The headgear strap magnet can either be sewn into one of the straps facing the hair or it can be removably attachable at any position to a strap.

FIG. 3 shows the patient wearing the assembly. The headgear is shown as having an upper and a lower strap, and the middle of the upper strap provides a magnetic coupling between the strap and the hair grip device 30.

FIG. 4 shows a first design of the headgear strap, in which the magnet 33 is bonded to a fabric base 34 which is then sewn onto the headgear strap 36.

FIG. 5 shows a second design in which the magnet 33 is attached to a base piece 38 of plastic or silicon with a loop fastening 40 on the bottom, such as Velcro (Trade Mark). The loop fastening allows for the movement of the magnet to different positions on the strap so that it can be placed anywhere where needed. Other securing arrangements can be used, such as poppers or a small belt which is provided around the headgear strap.

The hair grip device can be any known device which grips the hair, such as:
a hair comb (as shown);
a spring-closed butterfly clip;
a hair band which can for example be hooked onto a hook on the headgear strap;
a Kirby grip;
a curved plate metal sprung hair clip.
   Other hair grip devices can be used. The aim is to provide a hair grip that can be worn comfortably during sleep and which enables a coupling to a headgear strap.

In the examples above, the hair grip device is a standalone component, which is then releasably attached to a headgear strap. The releasable attachment can be magnetic as shown, or other connections can be used, such as poppers, hook and eye arrangements, Velcro fittings or an arrangement which is wrapped around the strap.

The hair grip device is preferably flat so that it is not uncomfortable when the patient lies their head back on top of the grip. Thus, a hair comb as shown or a curved plate spring hair clip may be of particular interest.

Instead of a separate item, the hair grip device can be incorporated into the headgear strap. For example it may have a hinged opening which is closed around the hair and clips shut on the headgear strap.

Thus, the invention is essentially to provide a coupling between a hair grip device and a headgear strap to fix the headgear strap relatively to the patient's hair. Various combinations of fixings are possible.

One or more hair grip devices may be used. These may be centrally positioned as shown or laterally positioned. They may be positioned to take account of the regions of thickest hair growth of the patient.

In the examples above, the hair grip couples a strap of the headgear to the hair of the patient. The coupling may be instead or additionally be on another part of the headgear, for example near the ears, before the separate straps are defined.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1. A patient interface assembly, comprising:
a patient interface device for communicating a gas to the nose and/or mouth of a patient;
headgear comprising a strap arrangement for securing the patient interface device against the face of the patient and having at least one strap; and
a hair grip device for coupling the headgear to the hair of the patient, wherein the hair grip device comprises a releasable coupling for releasably coupling the hair grip device to the headgear.

2. An assembly as claimed in claim 1, wherein the hair grip device is for coupling the at least one strap of the of the strap arrangement to the hair of the patient.

3.-4. (canceled)

5. An assembly as claimed in claim 4, wherein the releasable coupling comprises a magnetic element and the headgear is provided with a corresponding magnetic element.

6. An assembly as claimed in claim 5, wherein the magnetic element of the headgear is fixed in position to the headgear.

7. An assembly as claimed in claim 5, wherein the magnetic element of the headgear is removably mounted to the headgear.

8. An assembly as claimed in claim 1, wherein the headgear comprises a coupling for receiving the releasable coupling of the hair grip device, and the headgear coupling is removably mounted to the headgear.

9. An assembly as claimed in claim 1, wherein the hair grip device comprises a hair comb.

10.-11. (canceled)

12. A headgear assembly for securing a patient interface to a patient's head, comprising:
at least one strap; and
coupling means for releasable coupling of the headgear assembly to a hair grip device which is for coupling the headgear to the hair of the patient, wherein the hair grip device comprises a releasable coupling for releasably coupling to the coupling means.

13. (canceled)

14. The headgear assembly as claimed in claim 12, wherein the headgear assembly comprises the hair grip device.
